(19) United States
(12) Patent Application Publication
(10) Pub. No.: US 2004/0234509 A1
(43) Pub. Date: Nov. 25, 2004

(54) Replacing liver cells with bone marrow-derived cells for treating disease and expressing therapeutic genes

(76) Inventor: Roger A. Davis, Solana Beach, CA (US)

Correspondence Address: McDERMOTT, WILL & EMERY, 7th Floor, 4370 La Jolla Village Drive, San Diego, CA, 92122, US

(21) Appl. No.: 10/616,690

(22) Filed: Jul. 9, 2003

Related U.S. Application Data
(60) Provisional application No. 60/394,569, filed on Jul. 9, 2002.

(51) Int. Cl. A61K 45/00; A61K 9/127; A61K 33/24

(52) U.S. Cl. 424/93.7; 424/617; 424/450

(57) ABSTRACT

The invention provides a method for introducing a population of Kupffer cells into an individual. The method includes the steps of (a) administering to an individual a Kupffer cell toxin, wherein the toxin ablates a first population of Kupffer cells of the individual, and (b) administering to the individual a population of Kupffer cell progenitors, wherein the population of Kupffer cell progenitors replaces the first population of Kupffer cells, thereby providing a second population of Kupffer cells.

This application is based on, and claims the benefit of, U.S. Provisional Application No. 60/394,569, filed Jul. 9, 2002, which is incorporated herein by reference.

This invention was made with government support under grant number HL57974 awarded by the National Institutes of Health. The United States Government has certain rights in this invention.

### BACKGROUND OF THE INVENTION

This invention relates generally to methods of treating diseases with gene-based therapies and, more specifically to methods for repopulating tissues with cells that are derived from the bone marrow and either express therapeutic genes or lack the expression of disease-promoting genes.

Essentially all disease can be cured or ameleorated by changing the expression of one or more genes and/or by changing the function of enzymes. The acknowledgment that our understanding of human disease can be significantly augmented by identifying a responsible genetic aberration, has been a major impetus in efforts to sequence the human genome as well as the genomes of other organisms that provide models of human disease. The international effort to sequence the human genome is largely completed and has increased the ability to identify the genes that are responsible for the processes that either cause or block human disease. Thus, the therapeutic as well as disease-causing genes are being rapidly identified.

Diseases can be ameliorated by altering the expression of one or more genes. A therapeutic correction in gene expression can be achieved by increasing or decreasing expression in an individual to correct for levels of a gene product that are inappropriate for normal function. A correction can also be made to introduce a gene into an individual in order to compensate for a gene that has a mutation that either blocks the expression of its gene product or produces a gene product that lacks its normal function or has assumed an abnormal function. Therapeutic gene correction has been applied to a number of diseases including, for example, Adenosine Deaminase Deficient Severe Combined Immune Deficiency (ADA-SCID), hemophilia, cardiovascular disease and cancer. Similar genetic level manipulations are being used to target infectious diseases such as Acquired Immune Deficiency Syndrome (AIDS) for which a conventional therapy is not available.

One of the largest challenges to developing a therapeutic method for correcting a genetic disease or condition is that of delivering the therapeutic gene to its target cell. One of the most widely used approaches is to clone a corrective gene into a viral vector which delivers the gene to a target cell or tissue. Viral vectors are typically viruses that have been altered so that they lack disease causing components while maintaining components that still allow targeting to a cell so that the inserted gene they have been assembled to contain will be expressed. Careful precautions must be taken to ensure that the viruses cannot replicate and that they do not cause the expression of proteins that will lead to immunologic and inflammatory responses.

Typical gene therapy has been confined to two major methods: (1) administering a virus to an individual via intravenous injection or injection into a specific tissue with the expectation that the virus will be taken up mainly by the target tissue in a manner that will allow an efficaceous level of expression of a therapeutic gene; and (2) administering a virus containing the therapeutic gene to cells and then injecting these cells into a patient with the expectation that the cells will survive and/or replicate and allow the expression of the therapeutic gene. An example of the first method is the use of adenovirus vectors to administer factor VIII to hemophilia A mice (Connelly et al., “Evaluation of an adenoviral vector encoding full-length human factor VIII in hemophiliac mice,” Thromb Haemost, 81(2):234-239 (1999).) An example of the second method is the use of adenovirus vectors to express LDL receptors in liver cells obtained from HFH patients (Grossman et al., “Successful ex vivo gene therapy directed to liver in a patient with familial hypercholesterolaemia,” JM. Nat. Genet., 6(4):335-341 (1994)). Administration of bone marrow derived stem cells expressing therapeutic genes has been used to treat some forms of leukemia. Typically, patients undergoing treatment with bone marrow cells are first treated with radiation in order to destroy their population of bone marrow stem cells. This procedure helps to ensure that the administered bone marrow stems cells will prevail as the major cell type present in bone marrow. Unfortunately, the use of radiation usually leads to a severe loss of immunologic defense and thus susceptibility to other infections and diseases.

Thus, there exists a need for a method of delivering a therapeutic gene to a target cell such that delivery is specific and provides for expression of therapeutic levels of the gene product. A need also exists for methods of gene delivery that avoids lethal radiation. The present invention satisfies these needs and provides related advantages as well.

### SUMMARY OF THE INVENTION

The invention provides a method for introducing a population of progenitor cells into an individual. The method involves (a) administering to the individual an amount of gadolinium chloride effective to ablate a first population of resident cells of the individual, and (b) administering to the individual a population of progenitor cells, wherein cells of the population of progenitor cells replace resident cells of the first population of resident cells.

The invention also provides a method for introducing a population of Kupffer cells into an individual. The method involves (a) administering to an individual a Kupffer cell toxin, wherein the toxin ablates a first population of Kupffer cells of the individual, and (b) administering to the individual a population of Kupffer cell progenitors, wherein the population of Kupffer cell progenitors replaces the first population of Kupffer cells, thereby providing a second population of Kupffer cells.

The invention further provides a method for transiently introducing a population of resident macrophages, such as Kupffer cells, into an individual. The method includes the steps of (a) administering to an individual a resident macrophage toxin, wherein the toxin ablates a first population of resident macrophages of the individual; (b) administering to the individual a population of resident macrophage progenitors, wherein the population of resident macrophage progenitors replaces the first population of resident macrophages, thereby providing a second population of resident macrophages, and (c) administering to the individual a resident macrophage toxin, wherein the toxin kills the second population of resident macrophages and wherein a third population of resident macrophage progenitors replaces the second population of resident macrophages. In one embodiment, the resident macrophage progenitors can contain a nucleic acid that encodes a gene product, thereby providing a second population of resident macrophages that expresses an effective amount of the gene product to reduce a disease or condition.

The invention further provides a method for reducing a disease or condition. The method includes the steps of (a) administering to an individual a resident macrophage toxin, wherein the toxin kills a first population of resident macrophages of the individual, and (b) administering to the individual a population of resident macrophage progenitors containing a nucleic acid that encodes a gene product, wherein the population of resident macrophage progenitors replaces the first population of resident macrophages, thereby providing a second population of resident macrophages and expresses an effective amount of the gene product to reduce the disease or condition.

In addition the invention provides a method for stimulating an immune response against an antigen. The method includes the steps of (a) administering to an individual a resident macrophage toxin, wherein the toxin kills a first population of resident macrophages of the individual, and (b) administering to the individual a population of genetically modified resident macrophage progenitors containing a transgene that encodes the antigen, wherein the population of resident macrophage progenitors differentiates into a second population of resident macrophages, replaces the first population of resident macrophages and expresses an effective amount of the antigen to stimulate an immune response.

### BRIEF DESCRIPTION OF THE DRAWINGS

FIG. 1 shows fluorescent micrographs of liver sections obtained from C57BL/6 mice treated with saline (Panel A) or gadolinium chloride (Panel B) prior to administration of 2×10⁶ stem cells from TgN(ACTbEGFP)1Osb mice.

FIG. 2 shows fluorescent micrographs of liver sections obtained from C57BL/6 mice treated with gadolinium chloride prior to administration of 10×10⁶ (Panel A) or 100×10⁶ (Panel B) stem cells from TgN(ACTbEGFP)1Osb mice.

### DETAILED DESCRIPTION OF THE INVENTION

This invention provides methods for populating a tissue of an individual with progenitor cells. According to the methods, a population of cells, such as liver cells, can be ablated in an individual and subsequently cells can be administered to the individual such that they replace these cells. The replacement cells can express an endogenous or exogenous gene having any of a variety of desired effects on an individual such as treating a disease or condition, stimulating an immune response or altering a physiologic state. In one embodiment, cells can be genetically modified prior to being administered to an individual such that the replacement cells express a gene product that is deficient in the individual or, alternatively, inhibits expression or activity of an undesirable gene product. However, a cell need not be genetically manipulated prior to being administered to an individual. For example, cells from a donor individual that naturally express a therapeutic gene product can be administered. The therapeutic gene can be one that is missing in the recipient individual or it can be one that is present in the recipient individual. The administered cells can also lack expression of a gene, such that the cells replace cells that express a deleterious gene product.

The cells that are replaced by this method include Kupffer cells as well as other cell types that are derived from the bone marrow. Such cells can include endothelial cells, immature macrophages, cells derived from the same progenitor cells from which Kupffer cells are derived, and cell types that can be repopulated in a tissue or organ after administration of a Kupffer cell toxin such as gadolinium chloride.

In one aspect of the invention, the methods exploit properties of Kupffer cells that provide a number of advantages for delivery of a gene and efficacious expression of its encoded gene product. Because Kupffer cells are macrophages that are located in the sinusoids of the liver, thereby having direct contact with blood plasma, a gene product that is secreted from a replacement Kupffer cell is secreted into the blood and can be systemically delivered to an individual by passage through the circulatory system. Because the liver is a relatively large organ, constituting about 5% of body weight in an average individual, and the Kupffer cell population constitute about 5% to 20% of the liver, this population provides a large reservoir that can be replaced with cells having a desired function or gene. Thus, an individual can be treated by the methods of the invention with a gene product supplied at a high capacity.

The methods can be used for either long term or transient gene expression as desired. Relatively long term expression can be achieved because, unlike circulating monocyte/macrophages, which are replaced about every 2 months, Kupffer cells do not actively replicate and are replaced slowly, for the most part remaining in the liver for over a year. For example, in mice the half-life of Kupffer cells is about 6 months as described in Kennedy and Abkowitz, “Kinetics of central nervous system microglial and macrophage engraftment: analysis using a transgenic bone marrow transplantation model” Blood, 90:986-993 (1997). For shorter term expression, replacement Kupffer cells that express a particular gene can be ablated, for example, by administering a Kupffer cell toxin, whereupon the individual's native bone marrow stem cells will repopulate the liver with Kupffer cells and other cell types having similar progenitor cells.

The invention provides a method of delivering a therapeutic gene to patients in a manner that has the following characteristics and benefits: (1) the delivery is achieved using specific cell types that will not replicate thus reducing the chances for tumor formation; (2) provides for expression of therapeutic levels of the gene product for long duration and in a manner that can be reversed such that dosage of therapeutic gene can be controlled; (3) the use of lethal radiation, required for bone marrow replantation, can be avoided; (4) expression of large quantities of the therapeutic gene by use of a large number of cells as provided by the vast reservoir of cells present in the liver, which is the largest internal organ, for expressing a therapeutic gene at levels similar to endogenous genes, thereby avoiding inflammation caused by over-expression of gene products from a smaller number of cells; and (5) the expression of the therapeutic gene by cells that are in direct contact with blood plasma allowing the uptake and/or secretion of substances such as enzyme proteins within blood.

As used herein the term “resident,” when used in reference to a cell, is intended to mean a cell within a tissue or organ that is removed by a resident cell toxin, such as gadolinium chloride, and is subsequently replaced by a cell derived from the bone marrow. As used herein, the term “Kupffer cell” is intended to mean a cell within the liver that resides within the sinusoids, is removed by gadolinium chloride administered intravenously and is subsequently replaced by a cell derived from the bone marrow. A cell included in the term can have phagocytic activity typical of macrophages found in other tissues and plasma. A Kupffer cell can also have characteristics that may be pronounced such as uptake of oxidized LDL, as described in Van Berkel et al., J. Biol. Chem. 266(4):2282-9 (1991). Other examples of resident macrophages included alveolar macrophages which are located in the lung and macrophages that are localized to inflamed tissues such as atherosclerotic lesions or plaques.

As used herein, the term “resident cell toxin” is intended to mean a substance that ablates a resident cell. As such, the term “resident macrophage toxin” is intended to mean a substance that ablates a resident macrophage. Ablation can include induction of apoptosis, necrosis or any other process that results in death of the cell. A substance included in the term resident cell toxin can selectively ablate a resident cell, for example, a resident macrophage, such as a Kupffer cell, while sparing at least one other cell type such as a hepatic parenchymal cells. Selectivity can be the result of selective administration to the anatomical location where the cell resides; selective binding of the substance to the cell, or a component thereof; selective uptake of the substance by the cell; selective inhibition of the cell or a component thereof by the substance; or selective activation of a cell death pathway by the substance. A substance included in the term can ablate a resident macrophage, for example, by inducing apoptosis or necrosis. A substance included in the term can ablate by chemical activity or radioactivity. Examples of substances that selectively ablate resident cells, including resident macrophages include, but are not limited to, gadolinium chloride and clodronate.

As used herein, the term “Kupffer cell toxin” is intended to mean a substance that ablates a Kupffer cell as well as other hematopoetic lineage cells within a tissue or organ, such as the liver. Substances that can be used to ablate Kupffer cells include, for example, toxins coupled to ligands that are targeted to Kupffer cells via cell surface receptors. For example, diptheria toxin can be coupled to acetyl-LDL such that the toxin is targeted to Kupffer cells by interaction with the acetyl-LDL receptor. Antisense RNA can be delivered to Kupffer cells using selective targeting and large complexes known to be taken up by Kupffer cells such as glycans which are conjugated or attached to substances and/or chemicals that induce cell death via apoptosis or necrosis.

As used herein, the term “progenitor cell” is intended to mean a multipotent stem cell capable of residing in a location within a tissue or organ previously occupied by resident cells. Such a progenitor cell can be derived, for example, from bone marrow, circulating blood, umbilical cord blood, as well as other sources. As used herein, the term “resident macrophage progenitor” is intended to mean a cell that is capable of residing in a location within a tissue or organ previously occupied by resident macrophages or other hematopoetic lineage cells. A progenitor cell can be administered intravenously and/or within the peritoneal cavity. A progenitor cell, such as a bone marrow derived progenitor cell or resident macrophage progenitor, can replace a cell that was ablated by gadolinium chloride. Examples of a cell that can replace a resident cell, such as a resident macrophage, include a stem cell, monocyte, or other developmental progenitor of a resident macrophage or any other cell that can differentiate via the myeloid pathway into a resident macrophage. Other examples of a cell that can replace a resident cell, include a stem cell that can differentiate to a cell type present in the tissue or organ that it repopulates, such as a cell type present in the spleen, lung, or bone marrow. A cell included in the term can contain a naturally occurring genetic compliment or can be genetically modified. As used herein, the term “Kupffer cell progenitor” is intended to mean a cell that is capable of residing in a sinusoidal location within the liver previously occupied by Kupffer cells or other hematopoetic lineage cells of the liver or other organs.

As used herein, the term “differentiate” is intended to refer to the process whereby a cell acquires one or more structural or functional distinctions that facilitate the functions and structure of a specific tissue or organ.

As used herein, the term “autologous,” when used in reference to a cell being administered to an individual, is intended to mean a cell previously obtained from the individual or a cell derived from a cell previously obtained from the individual. A cell included in the term can contain a genetic composition that is identical to that of other cells in the individual. Alternatively, a cell included in the term can be genetically modified.

As used herein, the term “heterologous,” when used in reference to a cell being administered to an individual, is intended to mean a cell previously obtained from another individual.

As used herein, the term “genetically modified,” when used in reference to a cell, is intended to mean having a nucleic acid composition that is changed compared to the naturally occurring nucleic acid composition of the cell. A change in a nucleic acid included in the term can be an addition of one or more nucleotides in a polynucleotide sequence, deletion of one or more nucleotides in a polynucleotide sequence, or change in composition of one or more nucleotides in a polynucleotide sequence. Examples of changes included in the term are gene-knockout, gene-knockin or introduction of a transgene. As used herein the term “transgene” is intended to mean a nucleic acid sequence present in an organism that is non-naturally occurring in the organism or incorporated at a non-natural location of the organism's genome. A transgene can encode, for example, an mRNA, a ribozyme, a protein or an antisense RNA.

As used herein, the term “gene product” is intended to mean a polypeptide or nucleic acid that is expressed from a nucleic acid. Specific examples of polypeptide gene products include peptides, polypeptides or proteins encoded by structural genes. Specific examples of nucleic acid gene products include RNA, such as mRNA or antisense RNA; DNA and viral vectors.

As used herein, the term “inhibitor of a gene product” is intended to mean a molecule that reduces the amount or an activity of a macromolecule expressed from a nucleic acid. A molecule included in the term can be a gene product such as a polypeptide or protein, a nucleic acid such as a DNA or RNA, or a molecule produced or modified by a gene product. The reduction in the amount or activity of a macromolecule can be complete or partial. Reduction in the amount of a macromolecule can include decreased replication, transcription or translation, or increased degradation. Reduction in an activity of a macromolecule can occur, for example, by binding of an inhibitor to the macromolecule, depletion of a molecule that increases activity of the macromolecule or covalent modification of the macromolecule, so long as at least one activity of the macromolecule is decreased in response to the inhibitor. An inhibitor of a pro-atherogenic molecule is a molecule that, when in the presence of a pro-atherogenic molecule, reduces an activity of the pro-atherogenic molecule that is associated with the initiation or progression of atherosclerosis. An inhibitor that reduces an activity of a pro-atherogenic molecule can be, for example, a polypeptide having paraoxonase activity such as a PON1 gene product, or functional fragment thereof; polypeptide having cholesterol-7α-hydroxylase activity such as a CYP7A1 gene product, or functional fragment thereof; or polypeptide having apolipoprotein A1 activity such as an APOA1 gene product, or functional fragment thereof.

As used herein, the term “expression element” is intended to mean a nucleic acid sequence that regulates transcription or translation of a nucleic acid sequence. The term can include tissue or cell specific regulatory sequences. Examples of sequences that regulate transcription include, for example, inducible promoters, constitutive promoters, enhancers, silencers, response elements and the like. Examples of sequences that regulate translation include, for example, 5′ UTR (untranslated region) sequences. Accordingly, the term “regulate,” when used in reference to a nucleic acid encoding a polypeptide, is intended to refer to control of nucleic acid or polypeptide expression in a constitutive, suppressible or inducible manner.

A variety of tissue specific expression elements useful in a method of the invention are well known to those skilled in the art, and can be selected depending on the particular resident cells to be replaced in a method of the invention. As used herein, the term “macrophage-specific expression” is intended to mean transcription or translation of a nucleic acid in a macrophage. The term can include transcription or translation of a nucleic acid under the control of any expression element that is active in a macrophage including, for example, under the control of a tissue-specific expression element, constitutive expression element, or inducible expression element. Thus, the term can include transcription or translation under the control of an expression element that is active in one or more cell types, so long as expression occurs in a macrophage. Macrophage-specific expression can also occur when a macrophage is genetically modified in vitro to express an inhibitor of a pro-atherogenic molecule resulting in expression of a transgene in the macrophage.

As used herein the term “macrophage-specific expression element” is intended to mean a nucleic acid sequence that activates transcription or translation of a nucleic acid in a macrophage. The term can also include an expression element that displays relatively less expression in a non-macrophage cell. The term can include a class A scavenger receptor expression element described in Horvai et al., Proc. Natl. Acad. Sci. USA 92:5391-5395 (1995), Moulton et al., Mol. Cell. Biol. 14:4408-4418 (1994), Moulton et al., Proc. Natl. Acad. Sci. USA 89:8102-8106 (1992) and Wu et al., Mol. Cell. Biol. 14:2129-2139 (1994) including, for example, human class A scavenger receptor expression elements (GenBank accession No. M93189). For example, a scavenger receptor expression element can include a class A scavenger receptor promoter sequence extending from about −696 to about +46 base pairs from the major transcription start site of the SR gene; a class A scavenger receptor core promoter, which can include a sequence extending from about −245 to about +46 base pairs from the major transcription start site of the SR gene or a class A scavenger receptor enhancer, which can include sequences from about −4.1 to about −4.5 kb from the major transcription start site.

As used herein the term “reducing,” when used in reference to a disease or condition, is intended to mean lessening the extent of the disease or condition, susceptibility to the disease or condition or a symptom of the disease or condition. Lessening can include reversing the development or progression of a disease or condition or symptom thereof. When used in reference to atherosclerosis, the term can include lessening arterial plaque size, decreasing the opportunity of the plaque to rupture or allow formation of thrombosis. The term can also include, for example, lessening one or more pathological conditions or chronic complications associated with a disease or condition such as, in the case of atherosclerosis, angina, intermittent claudication, critical stenosis, thrombosis, aneurysm, or embolism. Reduced susceptibility is a lower probability or potential of being affected by a disease or condition. Being affected by a disease or condition can include displaying a symptom, diagnostic marker or characteristic of the disease or condition.

As used herein the term “inhibit” is intended to mean preventing or reducing an activity or process. The term can include the prophylactic treatment of an individual to guard from the occurrence of a disease or condition. The term can also include arresting the development or progression of the disease or condition. When used in reference to atherosclerosis, the term can include preventing or forestalling plaque formation, reduced systolic expansion, abnormally rapid wave propagation, or reduced elasticity of the affected arteries. The term can also include, for example, inhibiting or arresting the progression of one or more pathological conditions or chronic complications associated with the disease or condition such as, in the case of atherosclerosis, angina, intermittent claudication, critical stenosis, thrombosis, aneurysm, or embolism.

The invention provides a method for introducing a population of progenitor cells into an individual. The method includes the steps of (a) administering to an individual an amount of gadolinium chloride effective to ablate a first population of resident cells of the individual, and (b) administering to the individual a population of progenitor cells, wherein cells of the population of progenitor cells replace cells of the first population of resident cells.

The invention also provides a method for introducing a population of resident macrophages into an individual. The method includes the steps of (a) administering to an individual a resident macrophage toxin, wherein the toxin ablates a first population of resident macrophages of the individual, and (b) administering to the individual a population of resident macrophage progenitors, wherein the population of resident macrophage progenitors replaces the first population of resident macrophages, thereby providing a second population of resident macrophages.

The invention also provides a method for transiently introducing a population of resident macrophages into an individual. The method includes the steps of (a) administering to an individual a resident macrophage toxin, wherein the toxin ablates a first population of resident macrophages of the individual; (b) administering to the individual a population of resident macrophage progenitors, wherein the population of resident macrophage progenitors replaces the first population of resident macrophages, thereby providing a second population of resident macrophages, and (c) administering to the individual a resident macrophage toxin, wherein the toxin kills the second population of resident macrophages and wherein a third population of resident macrophage progenitors replaces the second population of resident macrophages. In one embodiment, the resident macrophage progenitors can contain a nucleic acid that encodes a gene product, thereby providing a second population of resident macrophages that expresses an effective amount of the gene product to reduce a disease or condition.

The invention further provides a method for introducing a population of Kupffer cells into an individual. The method includes the steps of (a) administering to an individual a Kupffer cell toxin, wherein the toxin ablates a first population of Kupffer cells of the individual, and (b) administering to the individual a population of Kupffer cell progenitors, wherein said population of Kupffer cell progenitors replaces the first population of Kupffer cells, thereby providing a second population of Kupffer cells.

In addition the invention provides a method for transiently introducing a population of Kupffer cells into an individual. The method includes the steps of (a) administering to an individual a Kupffer cell toxin, wherein the toxin ablates a first population of Kupffer cells of the individual; (b) administering to the individual a population of Kupffer cell progenitors, wherein the population of Kupffer cell progenitors replaces the first population of Kupffer cells, thereby providing a second population of Kupffer cells, and (c) administering to the individual a Kupffer cell toxin, wherein the toxin kills the second population of Kupffer cells and wherein a third population of Kupffer cell progenitors replaces the second population of Kupffer cells.

A resident macrophage used in a method of the invention can be a Kupffer cell which is a resident macrophage of the liver. Kupffer cells can display high phagocytic activity and can remove from blood plasma particulate materials and microbes. Kupffer cells and liver endothelial cells are responsible for the uptake and removal from blood plasma of oxidized LDL. Kupffer cells are the major hepatic source of proinflammatory cytokines such as TNF α and IL-1 α and IL-1β. Kupffer cells are resident macrophages of the liver that constitute 5-20% of the cells in the liver and are located in the sinusoids of the liver where they carry out functions such as phagocytosis of foreign particles, removal of endotoxins and other noxious substances, and modulation of immune responses. The blood passing through the portal vein comes into contact with a rich supply of Kupffer cells allowing them to efficiently scavenge bacteria from blood. The Kupffer cell uptake of bacteria and other pathogens helps to reduce invasion into the systemic circulation. This rich supply of Kupffer cells allows the liver to act as an effective filter of the hepatic blood that is often secondarily involved in infections and other systemic disorders. Thus, the Kupffer cells provide a relatively large population of cells for targeted replacement by the methods of the invention allowing treatment of liver diseases or conditions, and diseases or conditions that are treatable by delivery of gene products to the circulatory system.

Kupffer cells have a wide variety of phenotypes, depending upon their localized environment and stage of differentiation. Kupffer cells can be identified based on their expression of characteristic genes which include: MOMA-2, TNFα, IL-1 α, IL-1 β or UPC2. An important side effect of uptake by Kupffer cells of some bacterial products, such as endotoxin, is the activation and secretion of inflammatory cytokines. These cytokines act to promote a protective response to bacterial invasion. However, associated with this response is inflammation. Chronic inflammation can result in hepatitis, cirrhosis and metabolic abnormalities such as lipidemia and insulin resistance.

Resident macrophages in an individual can be ablated by administering a resident macrophage toxin. The toxin can be resident macrophage-selective having a greater toxic effect on resident macrophages than other cells of the same tissue. In the case of Kupffer cells, a selective toxin will have a greater effect on Kupffer cells than other liver cells, including, for example, parenchymal cells. An example of a resident macrophage-specific toxin is gadolinium chloride. When intravenously administered, gadolinium chloride forms a particulate which is selectively taken up by resident macrophages and induces apoptosis of the cells and removal of the cells from the liver as described for example in Mizgerd et al., J. Leukoc. Biol. 59:189-195 (1996) and Adachi et al., Hepatol. 20:453-460 (1994). Because gadolinium chloride is specific for resident macrophages, such as Kupffer cells and alveolar macrophages, circulating monocytes and circulating macrophages remain viable following administration. In addition, bone marrow stem cells remain viable and competent such that replacement resident macrophages are localized to the appropriate tissue or organ. For example, replacement Kupffer cells are localized to the liver by about one week following gadolinium chloride treatment as described for example in Hardonk et al., J. Leukoc. Biol. 52:296-302 (1992).

Another example of a resident macrophage-specific toxin is clodronate. Clodronate, which can be delivered into macrophages using liposomes as vehicles, induces apoptosis of the cells, as described, for example, in van Rooijen et al., J. Immunol. Methods 174:83-93 (1994); van Rooijen et al. J. Immunol. Methods, 193:93-99 (1996); and Trends in Biotech. 15: 178-185 (1997). Clodronate liposomes can be used to deplete macrophages in the liver (Kupffer cells) as well as other macrophage populations, including those in the spleen (different macrophage subpopulations), lung (alveolar but not interstitial macrophages), peritoneal cavity, lymph nodes, joints (phagocytic synovial lining cells) and testis. However, capillary walls can not be crossed by clodronate liposomes, such that circulating monocytes and macrophages can remain viable following administration. Clodronate liposomes can be prepared as described, for example, in van Rooijen et al., J. Immunol. Methods 174:83-93 (1994).

A method of the invention can include a step of administering bone marrow derived progenitor cells to an individual such that the progenitors replace ablated resident cells. Examples of bone marrow derived progenitor cells include any cells contained in bone marrow capable of differentiating into cells resembling resident cells to be replaced in a manner that allows them to express functions characteristic of resident cells to be replace. In another embodiment, a method of the invention can include a step of administering resident macrophage progenitors to an individual such that the progenitors replace ablated resident macrophages. Examples of resident macrophage progenitors include any cells capable of differentiating into cells resembling resident macrophages in a manner that allows them to express functions characteristic of resident macrophages. Progenitor cells useful in a method of the invention include but are not limited to pluripotent hematopoietic stem cells, CFU-GEMM cells, CFU-GM cells, promonocytes, monocytes, or cultured cells that can be stimulated to differentiate into resident macrophages such as RAW 267.4 cells. The characteristics of each of these cell types that facilitate their identification and isolation, including relative size, density, granularity and presence of cell surface markers, are well known in the art (see, for example, Kuby, Immunology 3^{rd} ed., Freeman & Co., New York (1997)).

Prior to administration, progenitor cells can be treated with a substance that favorably affects a progenitor cell activity, such as progenitor cell differentiation or viability. For example, a macrophage progenitor cell can be treated with an antigen to obtain an activated macrophage progenitor cell capable of initiating an immune response.

Progenitor cells such as bone marrow derived progenitor cells, including resident macrophage progenitors, can be obtained can be obtained by methods known in the art, including density gradient separation through media such as Ficoll or Percoll, apheresis, and positive and negative selection methods (e.g. immunomagnetic selection or flow cytometry), alone or in any combination. Apheresis is a preferred method to remove large numbers of blood cells of a particular type (e.g. peripheral blood mononuclear cells or platelets) from an individual, while returning red blood cells. Cell separators suitable for apheresis and their uses are well known in the art, and include, for example, the FENWAL CS 3000™ cell separator (Baxter International Inc, Deerfield, Ill.), the HAEMONETICS MCS™ system (Haemonetics Corp., Braintree, Mass.), and the COBE Spectra Apheresis System™ (Gambro BCT). Another method of further selection of desired cell subsets is immunomagnetic selection using an automated cell selection system, such as an ISOLEX 300i™ cell selection device (Nexell Therapeutics Inc., Irvine Calif.).

A population of progenitor cells or resident macrophage progenitors can be administered to an individual before or after administration of a toxin depending upon the conditions of treatment. Under conditions where a resident cell toxin or resident macrophage-specific toxin is used and the toxin does not adversely affect the viability or differentiation of the progenitors, the progenitors can be administered before or after toxin. Alternatively, in those cases where the toxin has an effect on the progenitors, the timing and/or location of progenitor administration can be selected such that the progenitors do not come into contact with the toxin. For example, the progenitors can be administered following clearance of the toxin from the liver or at a site that sequesters or otherwise protects the progenitors from toxin prior to tissue or organ recolonization.

A population of progenitor cells, such as bone marrow derived progenitor cells or resident macrophage progenitors can be administered by intravenous injection as described for example in Example I. Alternatively cells can be administered to a particular tissue or organ such as the bone marrow or liver. The location for implantation can be chosen according to various other criteria including, for example, the presence of nutrients required for cell viability and the presence of growth factors or cytokines for differentiation of the cell. Accordingly, a monocyte or other resident macrophage progenitor can be implanted into the bone marrow of an individual such that maturation and release of the cells to the blood stream can occur by natural processes. Liver administration can be used for later stage Kupffer cell progenitors for which the environment of the liver favors viability or differentiation. Administration to a particular organ or tissue can be achieved by injection, surgical implantation or endoscopic delivery using methods known in the art.

One characteristic that can be exhibited by a population of progenitor cells, such as resident macrophage progenitors, to be administered is that they are substantially immunologically compatible with the recipient individual. A cell is immunologically compatible if it is either histocompatible with recipient host antigens or if it exhibits sufficient similarity in cell surface antigens so as not to elicit an effective host anti-graft immune response. Specific examples of immunologically compatible cells include autologous cells isolated from an individual to be treated and allogeneic cells which have substantially matched major histocompatibility (MHC) or transplantation antigens with the recipient individual. Immunological compatibility can be determined by antigen typing using methods well known in the art. Using such antigen typing methods, those skilled in the art will know or can determine what level of antigen similarity is necessary for a resident macrophage progenitor to be immunologically compatible with a recipient individual.

In addition to selecting progenitors that exhibit characteristics that maintain viability following administration to a recipient individual, methods well known in the art can be used to reduce the severity of an anti-graft immune response. Such methods can therefore be used to further increase the in vivo viability of immunologically compatible progenitors, such as resident macrophage progenitors, or to allow the in vivo viability of less than perfectly matched resident macrophage progenitors or of non-immunologically compatible resident macrophage progenitors. Therefore, for therapeutic applications, it is not necessary to select a cell type from the individual to be treated in order to achieve viability of the modified cell following administration. Instead, and as described further below, alternative methods can be employed which can be used in conjunction with essentially any donor resident macrophage progenitor to confer sufficient viability to achieve a particular therapeutic effect.

For example, in the case of partially matched or non-matched resident macrophage progenitors, immunosuppressive agents can be used to render the host immune system tolerable to administration of the cells. The regimen and type of immunosuppressive agent to be administered will depend on the degree of MHC similarity between the modified donor resident macrophage progenitor and the recipient. Those skilled in the art know, or can determine, what level of histocompatibility between donor and recipient antigens is applicable for use with one or more immunosuppressive agents. Following standard clinical protocols, administration and dosing of such immunosuppressive agents can be adjusted to improve efficiency of resident macrophage replacement and the viability of the cells of the invention. Specific examples of immunosuppressive agents useful for reducing a host anti-graft immune response include, for example, cyclosporin, corticosteroids, and the immunosuppressive antibody known in the art as OKT3.

A progenitor cell, such as a resident macrophage progenitor, that is administered to an individual in a method of the invention can have one or more naturally occurring genes or other characteristic that provide a desired effect on the individual. For example, a recipient individual having a genetic defect characterized by an absent or deficient gene can be administered a progenitor that was obtained from a donor having the gene, thereby correcting the genetic defect. Alternatively, a recipient individual having a gene that is expressed to the detriment of the recipient can be administered progenitor cells, such as macrophage progenitors, that are obtained from an individual lacking the offending gene.

A population of macrophage progenitors that is administered to an individual in a method of the invention can be modified to introduce a desired function to a progenitor cell or to remove an unwanted function. For example, a macrophage progenitor can be genetically modified to contain a transgene. A transgene that is contained in a macrophage progenitor can express a gene product that is deficient in a recipient individual or that provides an activity that is otherwise desired. A transgene can be introduced to a resident macrophage progenitor using methods known to those skilled in the art as described, for example, in Sambrook et al., Molecular Cloning: A Laboratory Manual, 2nd ed., Cold Spring Harbor Press, Plainview, N.Y. (1989); Sambrook et al., Molecular Cloning: A Laboratory Manual, 3rd ed., Cold Spring Harbor Press, Plainview, N.Y. (2001); Ausubel et al. (Current Protocols in Molecular Biology (Supplement 47), John Wiley & Sons, New York (1999)).

Suitable expression vectors include those capable of expressing a transgene operatively linked to a regulatory sequence or element such as a promoter region or enhancer region that is capable of regulating expression of the transgene in a resident macrophage. For example, a vector of the invention can include a nucleic acid encoding an inhibitor of a pro-atherogenic molecule operationally linked to a tissue-specific expression element, such as a macrophage-specific expression element. Promoters or enhancers, depending upon the nature of the regulation, can be constitutive, suppressible or inducible. The regulatory sequences or regulatory elements are operatively linked to a nucleic acid such that the physical and functional relationship between the nucleic acid and the regulatory sequence allows transcription of the nucleic acid.

Any of a variety of inducible promoters or enhancers can be functionally attached to a nucleic acid encoding a gene product for expression of a transgene in a resident cell, such as a resident macrophage. An inducible system particularly useful for therapeutic administration utilizes an inducible promotor that can be regulated to deliver a level of therapeutic product in response to a given level of drug administered to an individual and to have little or no expression of the therapeutic product in the absence of the drug. One such system utilizes a Gal4 fusion that is inducible by an antiprogestin such as mifepristone in a modified adenovirus vector (Burien et al., Proc. Natl. Acad. Sci. USA, 96:355-360 (1999). Another such inducible system utilizes the drug rapamycin to induce reconstitution of a transcriptional activator containing rapamycin binding domains of FKBP12 and FRAP in an adeno-associated virus vector (Ye et al., Science, 283:88-91 (1999)). It is understood that any combination of an inducible system can be combined in any suitable vector, including those disclosed herein. Such a regulatable inducible system is advantageous because the level of expression of the therapeutic product can be controlled by the amount of drug administered to the individual or, if desired, expression of the therapeutic product can be terminated by stopping administration of the drug.

Vectors useful for therapeutic administration of a gene product can contain a regulatory element that provides tissue specific expression of an operatively linked transgene. In one embodiment, the invention provides a nucleic acid encoding a desired gene product linked to a sequence of a macrophage-specific expression element. A macrophage-specific expression element can be a macrophage-specific promoter such as a class A scavenger receptor promoter or a macrophage-specific enhancer such as a class A scavenger receptor enhancer. The expression elements can be used individually or in various combinations to suit a particular application of the methods. Class A scavenger receptor expression elements prevent expression of an operationally attached gene in macrophage progenitor cells such as monocytes and activate expression of the gene upon macrophage differentiation as described in Horvai et al., supra (1995). Class A scavenger receptor expression elements induce expression in the presence of macrophage colony-stimulating factor (M-CSF), granulocyte macrophage colony-stimulating factor (GM-CSF), and phorbol ester phorbol 12-myristate 13-acetate (PMA). A macrophage-specific expression element can be operationally linked to a sequence encoding any desired gene product according to known properties and orientations of the expression element. Cloning methods useful for linking two nucleic acid sequences are known in the art as described, for example, in Sambrook et al., supra (1989); Sambrook et al., supra (2001) and Ausubel et al., supra (1999)).

A transgene can be specifically expressed by resident macrophages of a recipient organ or tissue even if its promoter is not subject to tissue or cell-type specific expression. To do so, the transgene is delivered by known methods such as transfection, to resident macrophage progenitors. Cells are then transferred into recipients who have been treated with agents that ablated resident macrophages. Although not required, the resulting resident macrophages can be senescent such that the expression of transgene can be confined to resident macrophages. Examples of such promoters include the cytomegalovirus promoter which provides a high level of constitutive gene expression as well as the pancreatic beta-cell glucokinase promoter which provides a glucose responsive gene expression.

A transgene can be delivered into a resident cell, such as a resident macrophage progenitor, either in vivo or in vitro using suitable vectors well-known in the art. Suitable vectors include viral vectors such as retroviral vectors, adenovirus, adeno-associated virus, lentivirus, herpesvirus, as well as non-viral vectors such as plasmid vectors. Such vectors are useful for providing therapeutic amounts of a gene product (see, for example, U.S. Pat. No. 5,399,346, issued Mar. 21, 1995).

Viral based systems provide the advantage of being able to introduce relatively high levels of a heterologous nucleic acid into a resident macrophage progenitor. Suitable viral vectors for introducing a transgene into a resident macrophage progenitor are well known in the art. These viral vectors include, for example, Herpes simplex virus vectors (Geller et al., Science, 241:1667-1669 (1988)); vaccinia virus vectors (Piccini et al., Meth. Enzymology, 153:545-563 (1987)); cytomegalovirus vectors (Mocarski et al., in Viral Vectors, Y. Gluzman and S. H. Hughes, Eds., Cold Spring Harbor Laboratory, Cold Spring Harbor, N.Y., 1988, pp. 78-84)); Moloney murine leukemia virus vectors (Danos et al., Proc. Natl. Acad. Sci. USA, 85:6460-6464 (1988); Blaese et al., Science, 270:475-479 (1995); Onodera et al., J. Virol., 72:1769-1774 (1998)); adenovirus vectors (Berkner, Biotechniques, 6:616-626 (1988); Cotten et al., Proc. Natl. Acad. Sci. USA, 89:6094-6098 (1992); Graham et al., Meth. Mol. Biol., 7:109-127 (1991); Li et al., Human Gene Therapy, 4:403-409 (1993); Zabner et al., Nature Genetics, 6:75-83 (1994)); adeno-associated virus vectors (Goldman et al., Human Gene Therapy, 10:2261-2268 (1997); Greelish et al., Nature Med., 5:439-443 (1999); Wang et al., Proc. Natl. Acad. Sci. USA, 96:3906-3910 (1999); Snyder et al., Nature Med., 5:64-70 (1999); Herzog et al., Nature Med., 5:56-63 (1999)); retrovirus vectors (Donahue et al., Nature Med., 4:181-186 (1998); Shackleford et al., Proc. Natl. Acad. Sci. USA, 85:9655-9659 (1988); U.S. Pat. Nos. 4,405,712, 4,650,764 and 5,252,479, and WIPO publications WO 92/07573, WO 90/06997, WO 89/05345, WO 92/05266 and WO 92/14829; and lentivirus vectors (Kafri et al., Nature Genetics, 17:314-317 (1997)).

For example, in one embodiment of the present invention, adenovirus-transferrin/polylysine-DNA (TfAdpl-DNA) vector complexes (Wagner et al., Proc. Natl. Acad. Sci., USA, 89:6099-6103 (1992); Curiel et al., Hum. Gene Ther., 3:147-154 (1992); Gao et al., Hum. Gene Ther., 4:14-24 (1993)) can be employed to transduce resident macrophage progenitors with a nucleic acid encoding a desired gene product.

A population of bone marrow derived progenitor cells, such as resident macrophage progenitors or Kupffer cell progenitors, that is administered into an individual can contain a transgene that expresses an inhibitor of a pro-atherogenic molecule. The inhibitor of a pro-atherogenic molecule can be selected from the group consisting of a paraoxonase polypeptide, cholesterol-α-hydroxylase polypeptide, apolipoprotein A1, or a functional fragment thereof. A transgene can include the nucleotide sequence of an inhibitor of a pro-atherogenic molecule such as any paraoxonase polypeptide including, for example, gene products of PON1 (Li et al., Pharmacogenomics 7:137-144 (1997)), PON2 (Mochizuki et al., Gene 213:149-157 (1998)) or PON3 (Reddy et al., Arterioscler. Thromb. Vasc. Biol. 21:542-547 (2001) and Draganov et al., J. Biol. Chem. 275:33435-33442 (2000)). For example, a nucleic acid molecule of the invention can include the sequence of the human PON1 cDNA (GenBank accession No. XM_{—}004948), or a fragment thereof. A nucleic acid encoding a PON1 polypeptide includes sequences that are the same or substantially the same as Accession No: XM_{—}004948. Other nucleic acid molecules encoding paraoxonase polypeptides useful in the invention include, for example, the sequence of the human PON2 cDNA (GenBank accession No. XM_{—}004947), the sequence of the mouse PON3 cDNA (GenBank accession No. NM_{—}008897), or a fragment thereof.

Other gene products that can be expressed from a transgene in a method of the invention include for example, D-glucosyl-N-acylsphingosine glucohydrolase to treat Gaucher's disease; insulin to treat diabetes; erythropoietin to treat anemia; factor VIII to treat hemophilia; antigens to act as vaccines in order to induce the production of antibodies, binding proteins to various cytokines and hormones in order to prevent their bioactivity such as binding proteins that bind to and block the ability of immune modulators such as TNF α to induce an inflammatory response.

A method of the invention can be used to introduce a population of resident cells, such as resident macrophages, into an individual that is a human. Alternatively, the individual can be any non-human mammal such as a mouse, including particular strains described herein, a rabbit, goat, pig, guinea pig, sheep, cow, or non-human primate. As set forth below, a method of the invention can be used for therapeutic treatment of an individual. A method of the invention can also be used to create a non-human mammal for testing the effects of various treatments or therapies when a particular gene is expressed by a resident macrophage. A method of the invention can also include expressing proteins in animals for the purpose of changing the animal's physiology including, for example, administering somatostatin to increase milk production in dairy cows or administering growth hormone to increase growth rate of juvenile animals.

The invention further provides a method for reducing a disease or condition. The method includes the steps of (a) administering to an individual a Kupffer cell toxin, wherein the toxin kills a first population of Kupffer cells of the individual, and (b) administering to said individual a population of Kupffer cell progenitors containing a nucleic acid that encodes a gene product, wherein the population of Kupffer cell progenitors replaces the first population of Kupffer cells, thereby providing a second population of Kupffer cells and expresses an effective amount of the gene product to reduce the disease or condition.

The invention provides another method for transiently reducing a disease or condition. The method includes the steps of (a) administering to an individual a Kupffer cell toxin, wherein the toxin kills a first population of Kupffer cells of the individual; (b) administering to the individual a population of Kupffer cell progenitors containing a nucleic acid that encodes a gene product, wherein the population of Kupffer cell progenitors replaces the first population of Kupffer cells, thereby providing a second population of Kupffer cells and expresses an effective amount of the gene product to reduce the disease or condition, and (c) administering to the individual a Kupffer cell toxin following the reduction in the disease or condition, wherein the toxin kills the second population of Kupffer cells, whereby a third population of Kupffer cell progenitors replaces the second population of Kupffer cells.

The invention provides a further method for reducing a disease or condition. The method involves (a) administering to an individual an amount of gadolinium chloride effective to ablate a first population of resident cells of the individual, and (b) administering to the individual a population of progenitor cells containing a nucleic acid that encodes a gene product, wherein the population of progenitor cells replaces resident cells of the first population, thereby providing a population of progenitor cells capable of expressing the gene product to reduce the disease or condition.

The invention further provides another method for reducing a disease or condition. The method includes the steps of (a) administering to an individual a resident macrophage toxin, wherein the toxin kills a first population of resident macrophages of the individual, and (b) administering to the individual a population of resident macrophage progenitors containing a nucleic acid that encodes a gene product, wherein the population of resident macrophage progenitors replaces the first population of resident macrophages, thereby providing a second population of resident macrophages and expresses an effective amount of the gene product to reduce the disease or condition.

Administration of a resident macrophage toxin to an individual that contains a population of resident cells, such as resident macrophages, can be used to remove the resident cells, including replacement cells. Thus, a method of the invention can be used to provide expression of an introduced gene for a discreet, controllable period of time. The ability to control the time for which an introduced gene is expressed allows for treatment of transient conditions or those for which the presence of its gene product is desired for a discreet time period. A further advantage of being able to ablate replacement resident macrophages is that, if the replacement cells cause unwanted side effects they can be readily removed.

Replacement resident macrophages that are removed by administration of a resident macrophage toxin can be in turn replaced by an individuals naturally occurring resident macrophage progenitors. Alternatively, a second population of resident macrophage progenitors can be administered to replace those resident macrophages which were derived from a previous administration of resident macrophage progenitors. Thus, an individual can be repeatedly dosed with a particular gene product by re-administration of resident macrophage progenitors containing the associated gene. Accordingly, if a gene present in resident macrophages from a first administration declines in effect prior to achieving a desired therapeutic goal, the gene can be reintroduced. A method that includes a reintroduction step can be used to alter the dose of a particular gene product by administering cells that express the gene at different levels until a desired effect is achieved.

In one embodiment, a method of the invention can be used to treat any individual at risk for developing atherosclerosis or presenting symptoms associated with atherosclerosis, thereby reducing atherosclerosis in the individual. Those skilled in the art will know or be able to determine risk factors for developing atherosclerosis including, for example, the presence of one or more gene or allele genetically associated with the condition, diet, or level of physical activity. The particular combination of symptoms and/or risk factors that identify an individual to be treated by the methods can differ. For example, although high blood cholesterol can identify an individual at risk for developing atherosclerosis, an individual having a particular atherosclerosis associated allele can be at risk for developing the condition even when cholesterol levels are within a range considered normal for the general population. The appropriate symptoms and/or risk factors for identifying a particular individual to be treated by the methods of the invention can be readily determined by those skilled in the art.

Atherosclerosis can be reduced by administering to an individual a population of resident macrophage progenitors containing a nucleic acid that encodes an inhibitor of a pro-atherogenic molecule. The progenitors will localize to the site of an e=atherosclerotic lesion or plaque, thereby providing a localized effect. An inhibitor of a pro-atherogenic molecule when expressed by a resident macrophage at the site of an atherosclerotic lesion is capable of preventing formation of foam cells as well as smooth muscle cell growth. A reduction in atherosclerosis achieved by using a method of the invention can include, for example, lessening plaque size, increasing systolic expansion, normalizing wave propagation, or increasing elasticity of affected arteries.

A method of the invention can be used to treat any individual at risk for developing Gaucher disease or presenting symptoms associated with Gaucher disease. Gaucher disease can be reduced by administering to an individual a population of resident macrophage progenitors containing a nucleic acid that encodes D-glucosyl-N-acylsphingosine glucohydrolase.

A method of the invention can be used to treat any individual at risk for developing diabetes or presenting symptoms associated with diabetes. Diabetes can be reduced by administering to an individual a population of resident macrophage progenitors containing a nucleic acid that encodes insulin.

A method of the invention can be used to treat any individual at risk for developing inflammation or presenting symptoms associated with inflammation. Inflammation can be reduced by using the method to replace the patient's endogenous resident macrophages with macrophages that lack functioning lipoxygenases. For example, following ablation of a patient's resident macrophages, a population of resident macrophage progenitors which lack the ability to express the proteins that have been identified as having the enzymatic activity attributed to 12/15 lipoxygenase and/or 5-lipoxygenase can be administered. As a result of this replacement, resident macrophages will have lost the ability to express 12/15-lipoxygenase and/or 5-lipoxygenase. The loss of 12/15-lipoxygenase has been shown to block the formation of atherosclerosis (Cyrus et al., “Absence of 12/15-lipoxygenase expression decreases lipid peroxidation and atherogenesis in apolipoprotein e-deficient mice,” Circulation, 103:2277-2282 (2001).) and loss of 5-lipoxygenase has been associated with decreased asthma (Leff, A., “Discovery of leukotrienes and development of antileukotriene agents,” Ann Allergy Asthma Immunol, 86:4-8 (2001).). In addition, a method of the invention can be used to prevent inflammation caused by activation of the Toll-like receptor 4, by replacing endogenous macrophages with those that have reduced expression of the Toll-like receptor 4. Inflammation can also be reduced by administering resident macrophage progenitors that have reduced expression of inflammatory cytokines compared to the resident macrophages they replace. Treatment of inflammation by altering cytokine expression and activity is described for example in Miyake et al., J. Biol. Chem. 275(29):21805-8 (2000).

The invention also provides a method for stimulating an immune response against an antigen. The method includes the steps of (a) administering to an individual a resident macrophage toxin, wherein the toxin kills a first population of resident macrophages of the individual, and (b) administering to the individual a population of genetically modified resident macrophage progenitors containing a transgene that encodes the antigen, wherein the population of resident macrophage progenitors differentiates into a second population of resident macrophages, replaces the first population of resident macrophages and expresses an effective amount of the antigen to stimulate an immune response.

The invention further provides a method for stimulating an immune response against an antigen. The method includes the steps of (a) administering to an individual a Kupffer cell toxin, wherein the toxin kills a first population of Kupffer cells of the individual, and (b) administering to the individual a population of genetically modified Kupffer cell progenitors containing a transgene that encodes the antigen, wherein the population of Kupffer cell progenitors differentiates into a second population of Kupffer cells, replaces the first population of Kupffer cells and expresses an effective amount of said antigen to stimulate an immune response. The method can also involve (c) administering to the individual a Kupffer cell toxin following the stimulation of the immune response, wherein the toxin kills the second population of Kupffer cells, whereby a third population of Kupffer cell progenitors replaces the second population of Kupffer cells.

For example, a cDNA encoding a specific portion of a viral protein can be inserted into a transgene that is then transfected into stem cell progenitors of resident macrophages. These progenitors can then be administered to recipients who have been subjected to resident macrophage depletion, as described above. The expression of the viral protein by the replacement resident macrophages will serve to increase the number of B and T cells recognizing the viral protein as an antigen as can be determined by measuring the titers of antibodies that are present in the plasma of recipients using a ELISA assay. After observing the desired titer of antibodies, the resident macrophage toxin can be administered in order to deplete the resident macrophages that express the viral protein.

The following examples are intended to illustrate but not limit the present invention.

### EXAMPLE I

### Kupffer Cell Replacement in Mice

This example demonstrates replacement of Kupffer cells in the liver of mice by treatment of the mice with gadolinium followed by administration of bone marrow cells to the mice.

Replacement of Kupffer cells in mice was demonstrated using stem cells from the bone marrow of C57BL/6-TgN(ACTbEGFP)1Osb mice which contain a transgene expressing GFP in every cell type, under the control of the actin promoter (Okabe et al., FEBS Letters. 407:313-319 (1997)). Bone marrow derived from C57BL/6-TgN(ACTbEGFP)10sb mice was injected into the tail vein of recipient C57BL/6 mice. Recipient mice were separated into two groups. One group received saline injection the other received gadolinium chloride (20-60 mg/kg i.v.: ICN Biomedicals, Aurora, Ohio) 24 hours before they received the bone marrow cells obtained from C57BL/6-TgN (ACTbEGFP) 10sb mice. The amount of bone marrow cells administered varied between 2×10⁶ to 100×10⁶ cells/animal. Mice were sacrificed 2 weeks after the stem cells were injected and livers were removed, fixed and prepared for fluorescent microscopy.

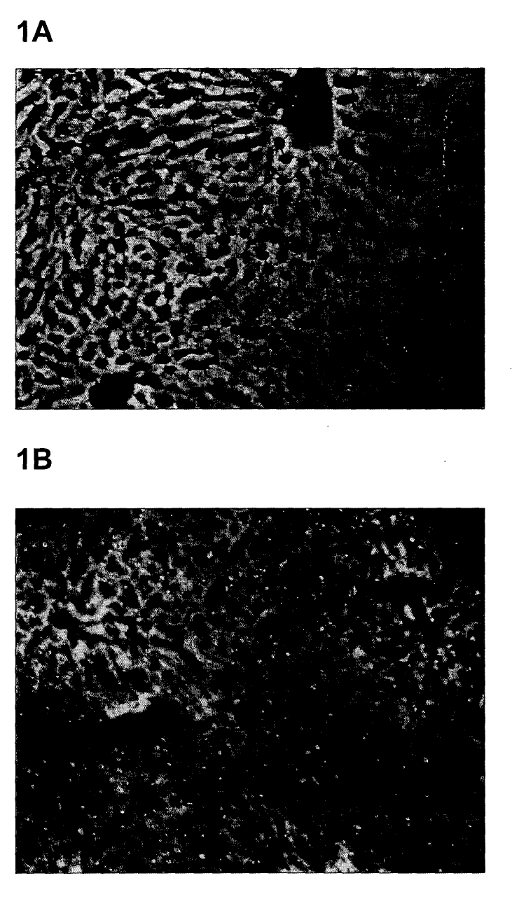

All mice survived and none showed evidence of any toxicity. The livers of C57BL/6 mice, that were injected with gadolinium chloride prior to stem cell administration, contained many GFP-fluorescent Kupffer cells while livers from control mice that received only the saline vehicle contained almost no GFP-fluorescent Kupffer cells. As shown by the representative micrographs in FIG. 1, GFP-fluorescent Kupffer cells were observed in the livers of mice injected with gadolinium chloride prior to stem cell administration as brightly lit spots (see panel B), whereas there were almost no bright spots in the Kupffer cells in livers from saline treated control mice (see panel A).

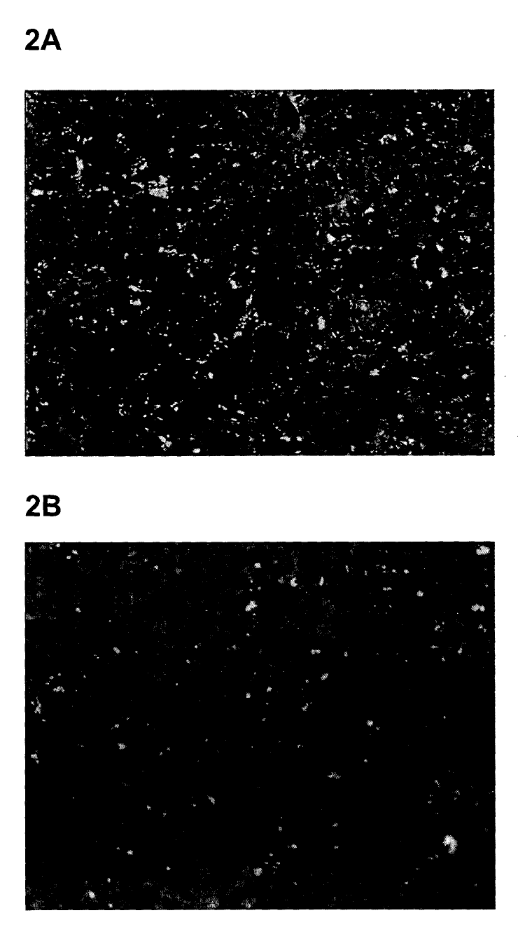

Administration of increased numbers of stem cells resulted in increased replacement of Kupffer cells in a saturable manner. C57BL/6 mice were injected with 60 mg/Kg of gadolinium chloride 24 hours before injecting stem cells intravenously. As shown in FIG. 2A, two weeks after injecting 10×10⁶ stem cells there were many more GFP labeled Kupffer cells in the livers of recipient mice than there were in the livers of mice receiving 2×10⁶ stem cells (FIG. 1B). Injecting more stem cells (100×10⁶ stem cells) did not significantly increase the number of GFP-labeled Kupffer cells (FIG. 2B) compared to injection of 10×10⁶ stem cells (FIG. 2A). These results suggest a finite capacity to replace Kupffer cells

Any disease for which the altered expression of proteins, enzymes or RNAs by a Kupffer cell can bring about an amelioration of the disease of the processes leading to the disease can be treated by a method of the invention. This method can be applied to treat any disease for which its diminution can be brought about due the augmentation of a chemical process that can either occur in blood plasma or can be carried out by transport into and out of the Kupffer cell. In addition, this method can be applied to treat any disease for which its diminution can be brought about due the blockage of a process whose origin is the Kupffer cell such as deletion of 12/15—as well as 5—lipoxygenases as described above).

Throughout this application various publications have been referenced. The disclosures of these publications in their entireties are hereby incorporated by reference in this application in order to more fully describe the state of the art to which this invention pertains.

The term “comprising” is intended herein to be open-ended, including not only the recited elements, but further encompassing any additional elements.

Although the invention has been described with reference to the examples provided above, it should be understood that various modifications can be made without departing from the spirit of the invention. Accordingly, the invention is limited only by the claims.

What is claimed is:

1. A method for introducing a population of progenitor cells into an individual, comprising the steps of:
(a) administering to an individual an amount of gadolinium chloride effective to ablate a first population of resident cells of said individual, and
(b) administering to said individual a population of progenitor cells,
wherein cells of said population of progenitor cells replace cells of said first population of resident cells.

2. The method of claim 1, wherein said first population of cells is contained in a tissue selected from liver, lung, spleen and bone marrow.

3. A method for introducing a population of Kupffer cells into an individual, comprising the steps of:
(a) administering to an individual a Kupffer cell toxin, wherein said toxin ablates a first population of Kupffer cells of said individual, and
(b) administering to said individual a population of Kupffer cell progenitors,
wherein said population of Kupffer cell progenitors replaces said first population of Kupffer cells, thereby providing a second population of Kupffer cells.

4. The method of claim 3, wherein said toxin comprises gadolinium chloride or clodronate liposomes.

5. The method of claim 3, wherein said Kupffer cell toxin is administered by intravenous injection.

6. The method of claim 3, wherein said population of Kupffer cell progenitors is administered by intravenous injection.

7. The method of claim 3, wherein said population of Kupffer cell progenitors comprises autologous cells obtained from said individual.

8. The method of claim 3, wherein said population of Kupffer cell progenitors comprises heterologous cells obtained from a donor individual.

9. The method of claim 3, wherein said population of Kupffer cell progenitors is genetically modified.

10. The method of claim 3, wherein said population of Kupffer cell progenitors is genetically modified to contain a transgene.

11. The method of claim 10, wherein said transgene expresses a macrophage gene product deficient in said individual.

12. The method of claim 11, wherein said gene product deficient in said individual comprises D-glucosyl-N-acylsphingosine glucohydrolase.

13. The method of claim 10, wherein said transgene expresses an inhibitor of a pro-atherogenic molecule.

14. The method of claim 13, wherein said inhibitor of a pro-atherogenic molecule is selected from the group consisting of a paraoxonase polypeptide, cholesterol-7α-hydroxylase polypeptide, apolipoprotein A1, or a functional fragment thereof.

15. The method of claim 10, wherein said transgene expresses a hormone.

16. The method of claim 15, wherein said hormone is selected from the group consisting of insulin and erythropoietin.

17. The method of claim 10, wherein said transgene comprises a macrophage-specific expression element.

18. The method of claim 17, wherein said macrophage-specific expression element comprises a macrophage-specific promoter or a macrophage-specific enhancer.

19. The method of claim 17, wherein said macrophage-specific expression element comprises a class A scavenger receptor promoter or enhancer.

20. The method of claim 3, wherein said population of Kupffer cell progenitors is modified to inhibit expression of a macrophage gene.

21. The method of claim 3, wherein said individual is a human.

22. The method of claim 3, wherein said individual is a non-human mammal.

23. A method for transiently introducing a population of Kupffer cells into an individual, comprising the steps of:
(a) administering to an individual a Kupffer cell toxin, wherein said toxin ablates a first population of Kupffer cells of said individual;
(b) administering to said individual a population of Kupffer cell progenitors,
wherein said population of Kupffer cell progenitors replaces said first population of Kupffer cells, thereby providing a second population of Kupffer cells, and
(c) administering to said individual a Kupffer cell toxin, wherein said toxin kills said second population of Kupffer cells and wherein a third population of Kupffer cell progenitors replaces said second population of Kupffer cells.

24. The method of claim 23, wherein said third population of Kupffer cells is administered to said individual.

25. A method for reducing a disease or condition, comprising the steps of:
(a) administering to an individual a Kupffer cell toxin, wherein said toxin kills a first population of Kupffer cells of said individual, and
(b) administering to said individual a population of Kupffer cell progenitors containing a nucleic acid that encodes a gene product,
wherein said population of Kupffer cell progenitors replaces said first population of Kupffer cells, thereby providing a second population of Kupffer cells and expresses an effective amount of said gene product to reduce said disease or condition.

26. The method of claim 25, wherein said disease comprises atherosclerosis.

27. The method of claim 26, wherein said gene product comprises an inhibitor of a pro-atherogenic molecule.

28. The method of claim 25, wherein said disease comprises Gaucher disease.

29. The method of claim 30, wherein said therapeutic gene product comprises D-glucosyl-N-acylsphingosine glucohydrolase.

30. The method of claim 27, wherein said disease comprises diabetes.

31. The method of claim 30, wherein said therapeutic gene product comprises insulin.

32. The method of claim 25, wherein said condition comprises inflamation.

33. The method of claim 32, wherein said transgene inhibits 12/15 lipoxygenase, 5-lipoxygenase, cytokine secretion or activation of Toll-like receptor 4.

34. A method for reducing a disease or condition, comprising the steps of:
(a) administering to an individual an amount of gadolinium chloride effective to ablate a first population of resident cells of said individual, and
(b) administering to said individual a population of progenitor cells containing a nucleic acid that encodes a gene product,
wherein said population of progenitor cells replaces resident cells of said first population, thereby providing a population of progenitor cells capable of expressing said gene product to reduce said disease or condition.

35. A method for transiently reducing a disease or condition, comprising the steps of:
(a) administering to an individual a Kupffer cell toxin, wherein said toxin kills a first population of Kupffer cells of said individual;
(b) administering to said individual a population of Kupffer cell progenitors containing a nucleic acid that encodes a gene product, wherein said population of Kupffer cell progenitors replaces said first population of Kupffer cells, thereby providing a second population of Kupffer cells and expresses an effective amount of said gene product to reduce said disease or condition, and
(c) administering to said individual a Kupffer cell toxin following said reduction in said disease or condition, wherein said toxin kills said second population of Kupffer cells, whereby a third population of Kupffer cell progenitors replaces said second population of Kupffer cells.

36. The method of claim 35, wherein said third population of Kupffer cells is administered to said individual.

37. A method for stimulating an immune response against an antigen, comprising the steps of:
(a) administering to an individual a Kupffer cell toxin, wherein said toxin kills a first population of Kupffer cells of said individual, and
(b) administering to said individual a population of genetically modified Kupffer cell progenitors containing a transgene that encodes said antigen,
wherein said population of Kupffer cell progenitors differentiates into a second population of Kupffer cells, replaces said first population of Kupffer cells and expresses an effective amount of said antigen to stimulate an immune response.

38. The method of claim 34, further comprising the step of:
(c) administering to said individual a Kupffer cell toxin following said stimulation of said immune response, wherein said toxin kills said second population of Kupffer cells, whereby a third population of Kupffer cell progenitors replaces said second population of Kupffer cells.
